(19) United States
(12) Patent Application Publication
(10) Pub. No.: US 2025/0243447 A1
(43) Pub. Date: Jul. 31, 2025

(54) 3D-PRINTED, WELL PLATE-INTEGRATED PIEZOELECTRIC CANTILEVER SENSORS FOR CELL CULTURE MONITORING

(71) Applicant: Virginia Tech Intellectual Properties, Inc., Blacksburg, VA (US)

(72) Inventors: Blake N. Johnson, Blacksburg, VA (US); Manjot Singh, San Marcos, CA (US)

(21) Appl. No.: 18/853,981

(22) PCT Filed: Apr. 4, 2023
(86) PCT No.: PCT/US23/17442

§ 371 Date: Oct. 3, 2024

Related U.S. Application Data
(60) Provisional application No. 63/327,089, filed on Apr. 4, 2022.

(51) Int. Cl. C12M 1/34; B33Y 10/00; B33Y 80/00; C12M 1/32; G01N 27/02; G01N 29/24; G01N 33/483

(52) U.S. Cl. CPC C12M 41/46; B33Y 10/00; B33Y 80/00; C12M 23/12; C12M 41/32; G01N 27/028; G01N 29/2437; G01N 33/4833

(57) ABSTRACT

A hybrid microextrusion pick-and-place 3D printing process enables fabrication of 3D-printed well plate-integrated piezoelectric cantilever sensors for monitoring properties and composition of tissue culture models. 3D-printed piezoelectric cantilever sensors exhibit multiple resonant modes across the 1-150 kHz frequency range. The ability to control cantilever frequency response (i.e., mode locations and types) was demonstrated by variation in the 3D-printed anchor geometry through corresponding changes in tool path and microextrusion process parameters. 3D-printed piezoelectric cantilever sensors facilitated in situ monitoring of cell culture media density and viscosity. This work presents an advance in the fabrication of cantilever sensors through the use of hybrid 3D-printing and pick-and-place processes that improves sensor reproducibility and enables novel control over anchoring of piezoelectric components and sensor integration with both 2D and 3D cell culture models, particularly 3D-bioprinted tissue constructs and microphysiological systems.

### STATEMENT OF GOVERNMENT INTEREST

This invention was made with government support under Grant No. CPS-1739318 awarded by the United States National Science Foundation. The government has certain rights in the invention.

### BACKGROUND OF THE INVENTION

### Field of the Invention

The present invention relates to the field of piezoelectric cantilever sensors. More specifically, the invention relates to processes for preparing such sensors and sensor arrays, such as in well plate formats, by way of a hybrid microextrusion pick-and-place 3D printing process, otherwise referred to as robotic embedding.

### Description of Related Art

Cell culture is central to various industries, including the agriculture, pharmaceutical, and healthcare industries. For example, the culture of animal, plant, and fungal cells is essential for the production of various products, including food, drugs, and fuels (Ramachandra Rao, S.; Ravishankar, G. A. Plant cell cultures: Chemical factories of secondary metabolites. Biotechnology Advances 2002, 20 (2), 101-153; Yue, W.; Ming, Q.-l.; Lin, B.; Rahman, K.; Zheng, C.-J.; Han, T.; Qin, L.-p. Medicinal plant cell suspension cultures: pharmaceutical applications and high-yielding strategies for the desired secondary metabolites. Critical Reviews in Biotechnology 2016, 36 (2), 215-232; Kantardjieff, A.; Zhou, W. Mammalian Cell Cultures for Biologics Manufacturing. In Mammalian Cell Cultures for Biologics Manufacturing; Zhou, W.; Kantardjieff, A., Eds.; Springer Berlin Heidelberg: Berlin, Heidelberg, 2014; pp 1-9.). 3D cell culture models, as opposed to monolayer culture models, have emerged as a critical aspect of animal cell culture driven heavily by applications in tissue and food engineering (Duval, K.; Grover, H.; Han, L.-H.; Mou, Y.; Pegoraro, A. F.; Fredberg, J.; Chen, Z. Modeling Physiological Events in 2D vs. 3D Cell Culture. Physiology 2017, 32 (4), 266-277; Tibbitt, M. W.; Anseth, K. S. Hydrogels as extracellular matrix mimics for 3D cell culture. Biotechnology and Bioengineering 2009, 103 (4), 655-663.).

Many cell culturing techniques still involve a lot of manual processing and offline quality control measures with small sample sizes. Most of the quality tests performed on cell cultures require long periods of time to process with large sample intervals. Manual control of these processes also leads to increased risk of error, hence decreasing quality and productivity. There is a need for on-line process monitoring and control with sensors embedded into the vessels used for the manufacturing of biologics and tissues. Continuous monitoring of processes with real-time control and high-throughput analysis will help eliminate some of the human related errors and help meet the tight regulatory standards of the industry.

Various types of sensor technologies such as optical, electrical impedance spectroscopy, electrochemical biosensors, and acoustic waves have been used to analyze cells and tissues. Zhu et al. used a microfluidic system in tandem with electrical impedance spectroscopy to enable subsequent culturing of immobilized S. pombe cells and continuous monitoring of dynamic changes in cell-cycle state and morphology over a broad frequency range. This system helped measure cell growth and clear impedance signals for nuclear division as well (Zhu, Z.; Frey, O.; Haandbaek, N.; Franke, F.; Rudolf, F.; Hierlemann, A., Time-lapse electrical impedance spectroscopy for monitoring the cell cycle of single immobilized S. pombe cells. Scientific reports 2015, 5, 17180-17180.). Several similar studies have used electrical impedance spectroscopy to monitor cell motility, mitosis, cell growth and motion (Zhu, Z.; Frey, O.; Franke, F.; Haandbwk, N.; Hierlemann, A. Real-time monitoring of immobilized single yeast cells through multifrequency electrical impedance spectroscopy. Analytical and Bioanalytical Chemistry 2014, 406 (27), 7015-7025; Ghenim, L.; Kaji, H.; Hoshino, Y.; Ishibashi, T.; Haguet, V.; Gidrol, X.; Nishizawa, M. Monitoring impedance changes associated with motility and mitosis of a single cell. Lab on a Chip 2010, 10 (19), 2546-2550.).

In another electrical impedance technique known as electric cell substrate impedance sensing (ECIS), a gold film electrode is placed at the bottom of the well such that attachment and proliferation of cells can be detected through impedance changes (Wegener, J.; Keese, C. R.; Giaever, I. Electric Cell-Substrate Impedance Sensing (ECIS) as a Noninvasive Means to Monitor the Kinetics of Cell Spreading to Artificial Surfaces. Experimental Cell Research 2000, 259 (1), 158-166.). The ECIS technique has also been integrated in a microfluidic device and used to measure cancer cell migration kinetics in a 3D matrix (Nguyen, T. A.; Yin, T.-I.; Reyes, D.; Urban, G. A. Microfluidic Chip with Integrated Electrical Cell-Impedance Sensing for Monitoring Single Cancer Cell Migration in Three-Dimensional Matrixes. Analytical Chemistry 2013, 85 (22), 11068-11076.). Other important quality parameters in a cell culture are dissolved oxygen (dO₂) and pH. In one study, researchers placed fluorescent sensors at the bottom of wells to continuously monitor pH and dissolved oxygen of the cell culture, which are indicative of cell growth and metabolic activity (Naciri, M.; Kuystermans, D.; Al-Rubeai, M. Monitoring pH and dissolved oxygen in mammalian cell culture using optical sensors. Cytotechnology 2008, 57 (3), 245-250, DOI: 10.1007/s10616-008-9160-1.). The properties discussed above are indicators of tissue quality. The dynamics of cell spreading, attachment, proliferation, migration, growth, viability, and metabolic activity plays a vital role in determining the quality of a tissue.

Another aspect in regenerative medicine is the use of lab grown tissues in various treatments, and it has been found that cells behave differently in 2D and 3D microenvironments. Hence, providing a native environment of extracellular matrix (ECM) is important to understand the cellular processes in bioprinted and engineered tissues. The microenvironment should be conducive for the cells to grow, proliferate and differentiate to form a specific tissue. Hydrogels have gained special attention for their abilities to mimic the ECM and to determine the fate of the stem cell while being modified by the stem cells during the differentiation process (Murphy, W. L.; McDevitt, T. C.; Engler, A. J. Materials as stem cell regulators. Nature materials 2014, 13 (6), 547-557.). Review of the literature has established the vital role that sensors can play in real-time in situ measurements of the cultures. Thus, the present inventors explored the application of 3D printing in integrating a sensor into common substrates for cell culture and bioprinted/molded tissues that could measure tissue quality attributes and material properties.

One of the critical challenges limiting progress in real-time monitoring of engineered tissues is sensor-process/product integration. Multi-material 3D printing and pick and place processes have recently emerged as useful fabrication methods for creating embedded electronics. For example, multi-material 3D printing processes have been used to fabricate microphysiological devices (Lind, J. U.; Busbee, T. A.; Valentine, A. D.; Pasqualini, F. S.; Yuan, H.; Yadid, M.; Park, S.-J.; Kotikian, A.; Nesmith, A. P.; Campbell, P. H.; Vlassak, J. J.; Lewis, J. A.; Parker, K. K., Instrumented cardiac microphysiological devices via multimaterial three-dimensional printing, Nature materials 2017, 16 (3), 303-308), chemical sensors (Siebert, L.; Lupan, O.; Mirabelli, M.; Ababii, N.; Terasa, M.-I.; Kaps, S.; Cretu, V.; Vahl, A.; Faupel, F.; Adelung, R., 3D-Printed Chemiresistive Sensor Array on Nanowire CuO/Cu2O/Cu Heterojunction Nets, ACS Applied Materials & Interfaces 2019, 11 (28), 25508-25515; Kennedy, Z. C.; Christ, J. F.; Evans, K. A.; Arey, B. W.; Sweet, L. E.; Warner, M. G.; Erikson, R. L.; Barrett, C. A., 3D-printed poly(vinylidene fluoride)/carbon nanotube composites as a tunable, low-cost chemical vapour sensing platform, Nanoscale 2017, 9 (17), 5458-5466), and biosensors (López Marizo, A. M.; Mayorga-Martinez, C. C.; Pumera, M., 3D-printed graphene direct electron transfer enzyme biosensors, Biosensors and Bioelectronics 2020, 151, 111980; Kadimisetty, K.; Mosa, I. M.; Malla, S.; Satterwhite-Warden, J. E.; Kuhns, T. M.; Faria, R. C.; Lee, N. H.; Rusling, J. F., 3D-printed supercapacitor-powered electrochemiluminescent protein immunoarray, Biosensors and Bioelectronics 2016, 77, 188-193). However, the integration of sensors with biomanufacturing processes and biological components remains a challenge.

### SUMMARY OF THE INVENTION

Embodiments of the invention include well plate-integrated 3D printed piezoelectric cantilever sensors and methods for producing said 3D printed piezoelectric cantilever sensors. Additional embodiments of the invention include methods for measuring mechanical properties of tissue or tumors using 3D printed piezoelectric cantilever sensors.

Specific aspects of embodiments of the invention include:

Aspect 1, which is a method for production of a 3D printed piezoelectric cantilever sensor comprising 3D printing of a sensor base/mold, optionally comprising silicone, dispensing epoxy within the sensor base/mold, placing one or more piezoelectric transducer, such as an insulated (e.g., spin coated or chemical vapor deposited) or uninsulated diced unimorph or bimorph piezoelectric (e.g. PZT) diced PZT chip with nickel electrodes into the epoxy, printing one or more, preferably at least two, conductive leads, such as conductive leads comprising conductive epoxy or colloidal inks, on one or more side/face of the piezoelectric cantilever, printing an asymmetric anchor on one side/face of the piezoelectric cantilever, wherein the asymmetric anchor is the same or a different material than the epoxy, dispensing a coating, such as a polyurethane coating, on one or more of the sensor base/mold, epoxy, piezoelectric cantilever and/or conductive leads, and curing the epoxy and/or coating to provide a piezoelectric cantilever sensor.

Aspect 2 is a method of Aspect 1, further comprising connecting one or more of the conductive lead(s) to an electrically-conductive circuit, such as one or more strips of conductive tape, for example comprising copper.

Aspect 3 is a method of Aspect 1 or 2, wherein the placing of the piezoelectric cantilever is performed using a tool with an L-shaped tip.

Aspect 4 is a method of any of Aspects 1-3, wherein the piezoelectric cantilever sensor is configured to be capable of measuring within a frequency of above 0 Hz to about 10 MHz, such as from about 100 Hz to about 100 kHz, or from about 100 kHz to about 1 MHz, or from about 10 kHz to about 500 kHz, or from about 1 kHz to about 150 kHz, or from above 0 kHz to about 100 kHz, or from about 100 kHz to about 200 kHz, or from about 50 kHz to about 1 MHz, or from about 1 kHz to about 250 kHz.

Aspect 5 is a method of any of Aspects 1-4, wherein the curing is performed at a temperature ranging from about 40-180° C., such as at a temperature of about 75° C.

Aspect 6 is a method of any of Aspects 1-5, wherein the curing is performed for a period of about 1 minute to about 3 hours, such as for about 15 minutes.

Aspect 7 is a method of any of Aspects 1-6, further comprising placing and/or securing one or more of the piezoelectric cantilever sensors into a well of a well plate, and optionally adding to one or more of the wells of the wall plate, any one or more of cells and/or engineered tissue, 3D printable and/or 3D printed cells, tissue and/or bioink, and/or cell culturing media and/or tissue engineering media.

Aspect 8 is a method of Aspect 7, wherein the piezoelectric cantilever sensor is constructed in a well plate.

Aspect 9 is a method of measuring mechanical properties of tissue or tumors comprising providing one or more piezoelectric cantilever sensor, disposing the piezoelectric cantilever sensor in a well plate with tissue or a tumor; and measuring one or more mechanical properties of the tissue or tumor with the piezoelectric cantilever sensor.

Aspect 10 is a method of Aspect 9, wherein at least part or all of the piezoelectric cantilever sensor is embedded in the tissue or tumor.

Aspect 11 is a method of Aspects 9 or 10, wherein one or more of the mechanical properties are measured using one or more electrical impedance measurements.

Aspect 12 is a method of any of Aspects 9-11, wherein one or more of the mechanical properties comprises stiffness of the tissue or tumor and/or mass changes in the tissue, the tumor and/or in cell culturing media or tissue engineering media.

Aspect 13 is a method of Aspect 12, further comprising measuring the stiffness and/or mass change of the tissue or tumor in response to one or more tissue or tumor treatment, such as chemotherapy.

Aspect 14 is a method of any of Aspects 9-11, further comprising monitoring one or more of cell motility, mitosis, cell growth, and/or motion and/or cell culture media density and/or viscosity.

Aspect 15 is a device comprising a well plate with one or more wells; and a piezoelectric cantilever sensor disposed in or capable of being placed in one or more of the wells, such as in each of the wells.

Aspect 16 is a device of Aspect 15, wherein the piezoelectric cantilever sensor is made by the method of any of Aspects 1-8.

Aspect 17 is a device of Aspect 15 or 16, wherein the piezoelectric cantilever sensor comprises a piezoelectric cantilever/chip with an asymmetric anchor on one face of the cantilever/chip.

Aspect 18 is a device of Aspect 17, wherein the cantilever/chip has a length of about 1 mm to about 5 mm.

Aspect 19 is a kit comprising one or more well plates with one or more wells; one or more piezoelectric cantilever sensor disposed in or capable of being placed in one or more of the wells, such as in each of the wells of one or more of the well plates; and optionally one or more of: cells and/or engineered tissue, 3D printable and/or 3D printed cells, tissue and/or bioink, and/or cell culturing media and/or tissue engineering media.

Aspect 20 is a method comprising: providing one or more piezoelectric cantilever sensor; disposing the piezoelectric cantilever sensor in a substance; and measuring mass or concentration of one or more analyte in the substance.

Aspect 21 is the method of Aspect 20, wherein the substance is a solution or crosslinked matrix.

Aspect 22 is a device comprising: a piezoelectric cantilever sensor with one or more microfluidic channels.

Aspect 23 is a batch of piezoelectric cantilever sensors comprising a cantilever/chip with a length of about 1 mm to about 5 mm, wherein each sensor in the batch (such as 10, 50, 100, up to 1,000 or more sensors in a batch) has a length of within 5-10% of a standard.

### BRIEF DESCRIPTION OF THE DRAWINGS

The accompanying drawings illustrate certain aspects of some of the embodiments of the present invention and should not be used to limit or define the invention. Together with the written description, the drawings serve to explain certain principles of the invention.

FIGS. 1A-H are illustrations showing fabrication steps for multi-material 3D-printed sensors, for example, by way of 3D printing and embedding (pick and place) processes.

FIG. 1I is a graph showing the phase angle vs. frequency with 3 modes visible with the asymmetric anchor and no mode visible on the sensor without asymmetric anchor.

FIG. 2A is an illustration showing an over-submergence defect in manual fabrication.

FIG. 2B is an illustration showing robotic fabrication with very low variation in the length and the angle.

FIG. 2C is a graph showing the difference in the length of the PZT cantilever anchored in epoxy when placed manually and robotically.

FIGS. 2D-F are illustrations showing the shape of the first three electrically measurable modes, where L_{cf}=Length of the cantilever−length of the asymmetric anchor

FIG. 3A is a graph showing spectra of a first mode of the sensor in various media along with their respective Q values.

FIG. 3B is a graph showing spectra of a second mode of the sensor in various media along with their respective Q values.

FIG. 3C is a graph showing spectra of a third mode of the sensor in various media along with their respective Q values.

FIGS. 4A-B are illustrations showing the printed cantilever sensors in 6-well plates.

FIG. 4C is a graph showing spectra of a first mode of the cantilever sensor from the 6 wells in air.

FIG. 4D is a graph showing spectra of a second mode of the cantilever sensor.

FIG. 5 is an illustration of a cantilever sensor interacting with a tissue model according to an embodiment of the invention.

FIG. 6 is an illustration of a cantilever sensor interacting with a tissue model comprising one or more microfluidic channels according to an embodiment of the invention.

FIG. 7 is an illustration of a tissue model interacting with a cantilever sensor according to an embodiment of the invention.

FIG. 8 is an illustration of a cantilever sensor interacting with a tissue model comprising one or more microfluidic channels according to an embodiment of the invention.

### DETAILED DESCRIPTION OF VARIOUS EMBODIMENTS OF THE INVENTION

Herein, provided is an additive manufacturing process for fabrication of piezoelectric cantilever sensors that facilitates improved sensor reproducibility, anchoring design, and sensor integration with cell culture platforms.

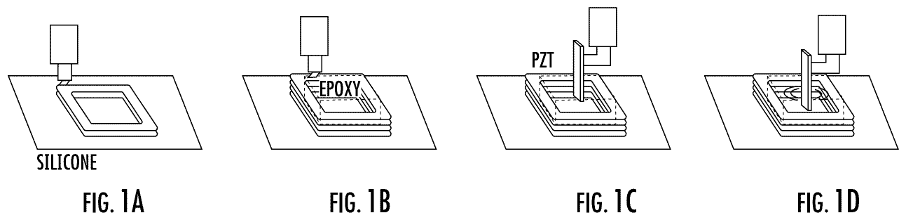

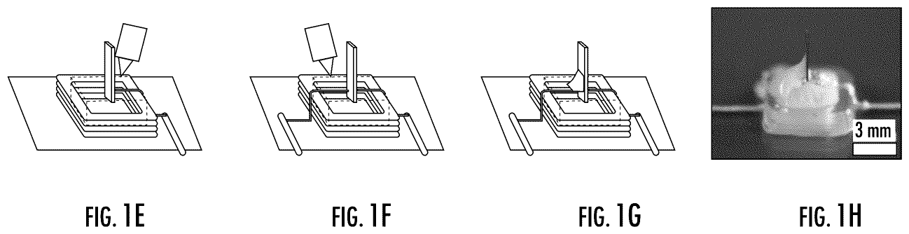

FIGS. 1A-H show the use of a multi-material microextrusion and pick and place process for fabricating piezoelectric cantilever sensors for their integration into common tissue culture environments. There are seven exemplary fabrication steps as shown in FIGS. 1A-H. FIG. 1A shows the printing of a hollow structure made of silicone (poly lactic acid, acrylonitrile butadiene styrene, or epoxy), which is followed by extrusion of epoxy (EA IC-LV) inside the silicone structure (FIG. 1B). FIGS. 1C-D show the pick and place process of the PZT, for example, lead zirconate titanate or Pb[Zr(x)Ti(1-x)]O₃ where 0≤x≤I, using an L-shaped tip. Negative pressure is applied on the nozzle which lifts the lead zirconate titanate (PZT) chip from the platform and places it in the epoxy mold. Printing is done on a heated stage and it takes around 10 minutes for epoxy to cure and harden. This step is followed by printing of silver conductive epoxy or other conductive inks (e.g., carbon nanotube based) on the distal ends of the opposite faces of the PZT and connected to the copper leads as shown in FIGS. 1E-F, which leads can be any conducting tape, wire or printed material. FIG. 1G shows the printing of the asymmetric anchor on one face of the PZT using epoxy (EA IC-LV). This is followed by coating of the sensor with polyurethane (e.g., manual coating) and curing it at room temperature to 75° C. in an oven for about 12-48 hours. FIG. 1H shows the final printed device.

FIG. 1I, shows the spectra of the sensor with asymmetric anchor and without asymmetric anchor. The printed sensors prior to the asymmetric anchor exhibited a purely capacitive response with no visible resonant modes but upon addition of the anchor, three electrically measurable modes were expressed between 1 kHz-150 kHz as depicted by the curve.

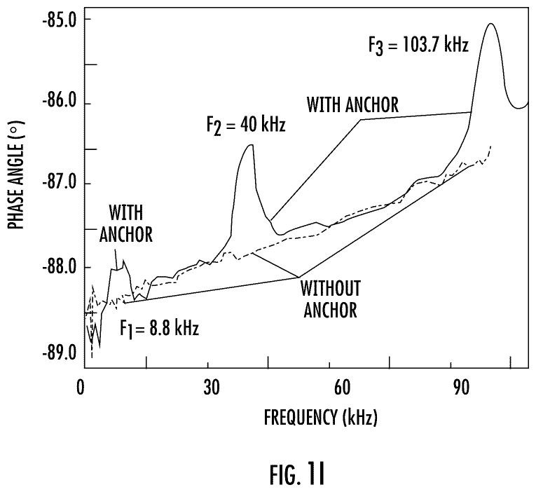

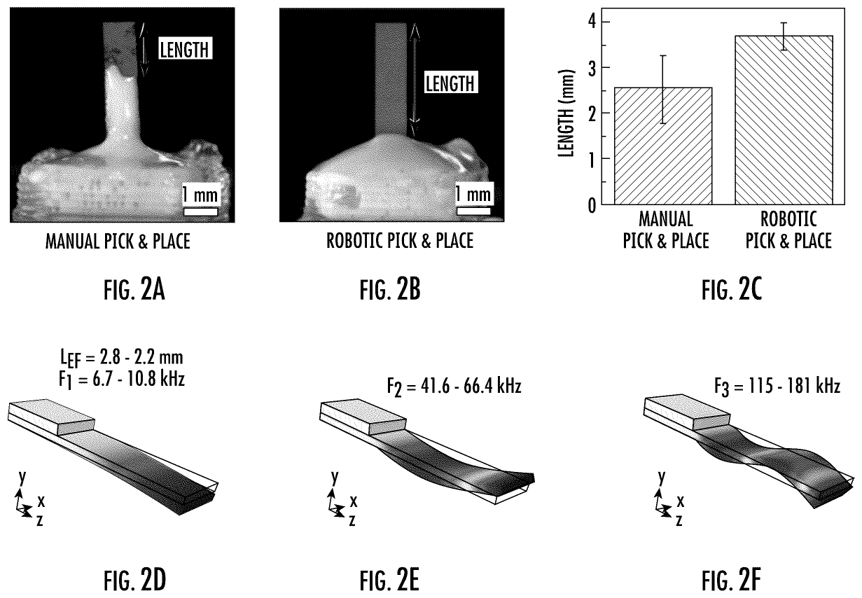

The multi-material 3D printing pick and place fabrication process was next compared with the manual fabrication process. The parameters affected by the fabrication process that also determine the sensor's response such as the resonant frequency, impedance, and the phase angle are the length (typically 2-4 mm) and the angle of the cantilever with the horizontal plane (typically 85-90°). It was found that with practice, hand-eye coordination improves and the angle of the vertical chip with the horizontal base in the manual fabrication was not statistically different than the angle in robotic fabrication but the length was significantly different. It was found to be difficult to hold the chip at the same length in the epoxy base throughout its curing period which led to over-submergence or submergence of the chip at the wrong length in epoxy, hence changing the length. The angles in manual fabrication and robotic fabrication were found to be 87.87°±1.57° and 88.33°±0.73° (p-value=0.74, n=10 samples), respectively, and the lengths in the manual fabrication and robotic fabrication were found to be 2.5 t 0.57 mm and 3.64 t 0.09 mm (p-value=0.0085, n=10 samples) respectively, with the target length as 3.8 mm (FIGS. 2A-C).

The value of the resonant frequency is dependent on the length of the cantilever chip and the length of the asymmetric anchor. FIGS. 2D-F show the shapes of the first three electrically measurable modes and dependence of resonant frequency on the effective length of the cantilever, which is the difference between the length of the cantilever and the length of the asymmetric anchor. As previously reported by Sharma et al., varying the effective length of the PZT chip with an asymmetric anchor brings changes in the resonant frequency as well as the phase angle of the resonant modes (Sharma, H.; Lakshmanan, R. S.; Johnson, B. N.; Mutharasan, R. Piezoelectric cantilever sensors with asymmetric anchor exhibit picogram sensitivity in liquids, Sensors and Actuators B: Chemical 2011, 153 (1), 64-70). Resonant frequency of a uniform cross-section cantilever depends on the material properties PZT and the length of the cantilever, as shown in Equation (1), where λₙ is the nth root of: 1+cos h(λₙ) cos (λₐ)=0; E is Young's modulus of PZT; I is the moment of inertia wt³/12. Resonant frequency is dependent on 1/L². Increasing the length of the asymmetric anchor decreases the length of the cantilever and hence resonant frequency increases. This is confirmed by the finite element studies as shown in FIGS. 2D-F.

$${f}_{n} = \frac{{λ}_{n}^{2}}{2 Π} \frac{1}{{L}^{2}} \sqrt{\frac{EI}{wt {ρ}_{c}}} \qquad Equation ( 1 )$$

Finite Element Analysis study was performed using COMSOL 5.3a. A parametric sweep of the anchor length was simulated using the eigenfrequency module. Simulations were run from extremely coarse to extremely fine mesh and error in the resonant frequency response was calculated. The error converged within 0.66% on the finer mesh. Hence, finer mesh was used for all the analysis. The fluid-structure interaction wasn't used and all the simulations were conducted in vacuum. The results of the finite element modeling show that with 3.8 mm as the length of the cantilever, if length of the asymmetric anchor is varied from 1 mm to 1.6 mm resonant frequency varies for first, second, and third mode from 6.7 kHz to 10.8 kHz, 416 kHz to 66.4 kHz, and 115 kHz to 181 kHz respectively. Multiple sensors were fabricated with 3.8 mm as the length of the cantilever and three electrically measurable modes were observed in the range closely matching the FEM simulation results.

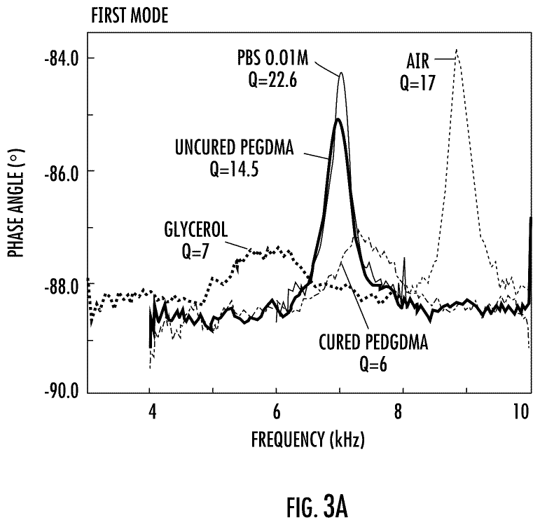

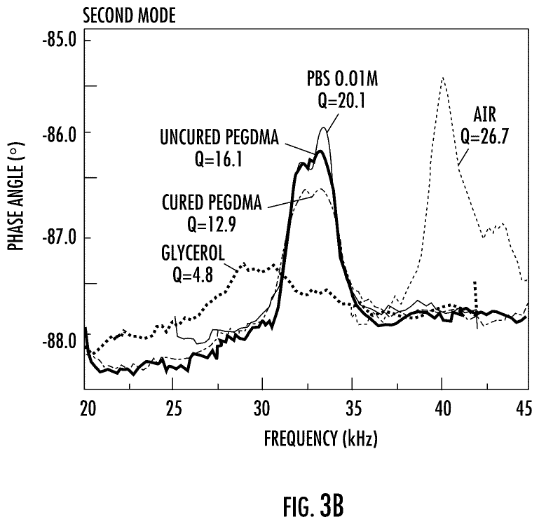

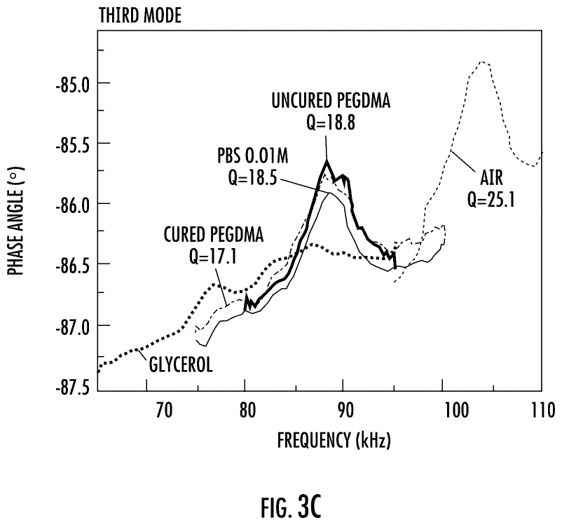

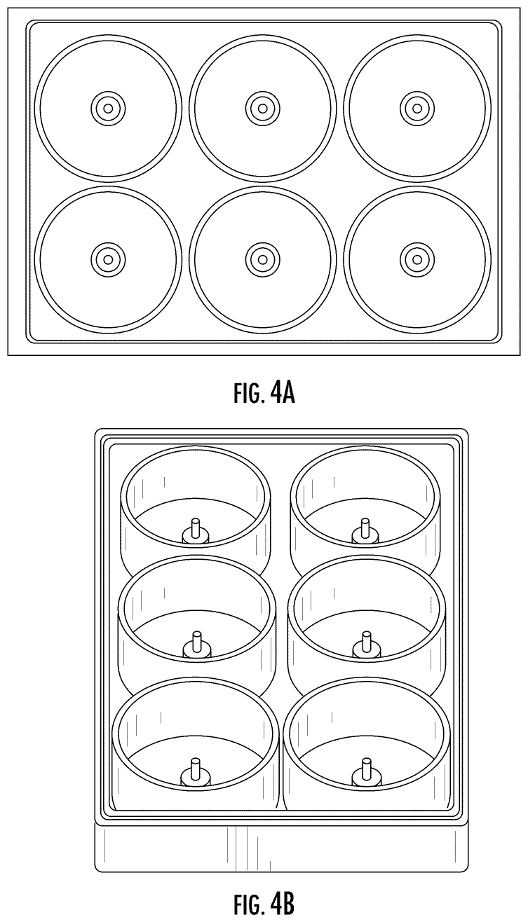

Having fabricated a cantilever sensor as shown in FIG. 1I, and comparing it with the manual fabrication process, its frequency response was then characterized (FIGS. 3A-C). A first resonant mode was expressed at 8.8 kHz (FIG. 3A), a second resonant mode was at 40.1 kHz (FIG. 3B), and a third mode at 103.7 kHz in air (FIG. 3C). Next, the behavior of these sensor signals was examined in different media which are important for cell culturing and tissue engineering. The sensor was submerged in PBS 0.01 M, PEGDMA solution, UV crosslinked PEGDMA, and glycerol. The values observed at the first mode in air and PBS are as follows: fₐᵢᵣ=8.84 kHz, Φₐᵢᵣ=−83.77°, Q-factorₐᵢᵣ=17, and Zₐᵢᵣ=23.675, f_{PBS}=7.02, Φ_{PBS}=−84.18, Q-factor_{PBS}=22.65, and Z_{PBS}=29.873. The change in resonant frequency and phase angle from air to PBS is −1.82 kHz and −0.41°. Similarly, the change in resonant frequency and phase angle from air to photopolymerized PEGDMA hydrogel is −1.54 kHz and −3.25°. Similar results were found in the results of second mode with Δf_{air-PBS}=−6.7 kHz, ΔΦ_{air-PBS}=−0.49°, Δf_{air-PEGDMA}=−6.9 kHz, ΔΦ_{air-PEGDMA}=−1.06°. This shows that the cantilever sensor is sensitive to mass change and stiffness changes in the media surrounding it. The second mode seems to be more sensitive than the first mode. The changes in the resonant frequency can be explained by Equation (2):

$$Δ f = \frac{1}{2} {f}_{n} ( \frac{Δ k}{k} - \frac{Δ m}{{m}_{n}} ) \qquad Equation ( 2 )$$

This expression shows that the resonant frequency is dependent on the mode of the resonance and it decreases with the increase in the mass, increases with the increase in the spring constant (Johnson, B. N.; Mutharasan, R., Biosensing using dynamic-mode cantilever sensors: A review, Biosensors and Bioelectronics 2012, 32 (1), 1-18).

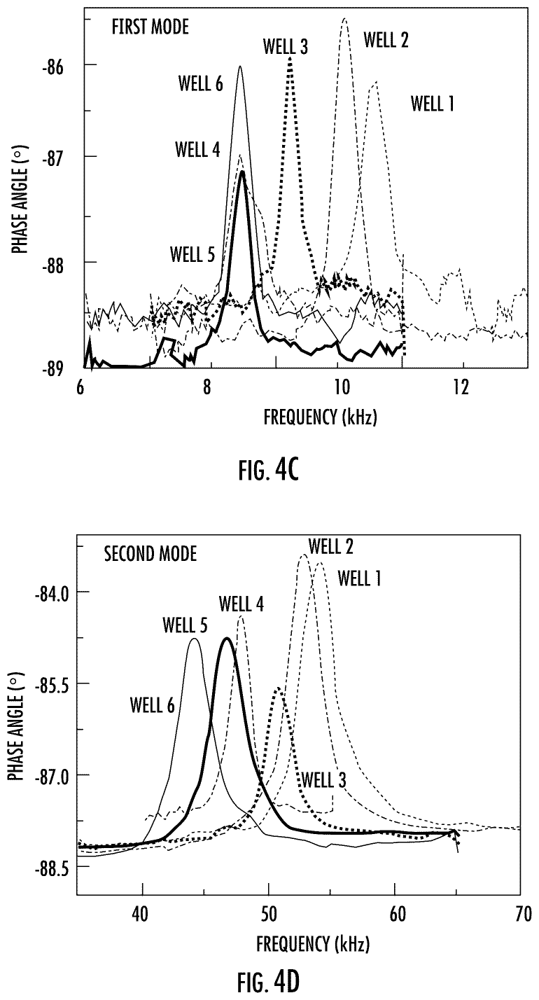

Having established that cantilever sensors can be manufactured using a multi-material 3D printing approach with pick and place capability and characterize them in different media, the substrate was changed to a common substrate for tissue fabrication to check the feasibility of a smart culture plate. FIGS. 4A-B show the sensors printed in an exemplary 6-well plate. FIGS. 4C-D show the spectra associated with the sensors printed in the exemplary 6-well plate for the first (FIG. 4C) and second (FIG. 4D) mode of the sensor.

After validating the sensor fabricated inside a culture plate, it was examined whether cantilever sensors exhibited resonance in complex matrices such as cell culture medium and hydrogels, that form the basis of tissue culture/engineering.

TABLE 1
Summary of PEMC frequency response characteristics including resonant frequency, phase angle at resonance (f),
quality factor (Q), and impedance at resonance (Z) (standard deviations based on a batch of n = 3 sensors).

| | f (kHz) | | Φ at Resonance (degrees) | | Q-factor | | Z (kOhm) | |
|---|---|---|---|---|---|---|---|---|
| | I Mode | II Mode | I Mode | II Mode | I Mode | II Mode | I Mode | II Mode |
| Air | 9.76 ± 1.96 | 46.82 ± 6.32 | −84.02 ± 0.75 | −85.18 ± 0.71 | 23.20 ± 6.02 | 26.08 ± 3.88 | 22.38 ± 3.8 | 4.73 ± 0.56 |
| PBS 0.01M | 8.02 ± 1.5 | 38.94 ± 5.44 | −84.76 ± 0.86 | −86.01 ± 0.65 | 24.19 ± 5.62 | 15.66 ± 6.5 | 26.76 ± 3.9 | 5.65 ± 0.73 |
| Uncured PEGDMA | 7.7 ± 1.79 | 38.5 ± 5.87 | −85.19 ± 0.46 | −85.69 ± 0.39 | 14.92 ± 2.2 | 18.70 ± 4.24 | 29.18 ± 6.12 | 5.95 ± 1.17 |
| Cured PEGDMA | 8.05 ± 1.80 | 38.53 ± 5.84 | −87.11 ± 0.57 | −86.14 ± 0.42 | 7.77 ± 1.83 | 15.33 ± 3.26 | 28.4 ± 6.03 | 5.97 ± 1.18 |
| Glycerol | 7.12 ± 1.58 | 34.52 ± 5.45 | −87.36 ± 0.04 | −87.20 ± 0.23 | 8.28 ± 1.84 | 6.30 ± 1.51 | 30.71 ± 6.17 | 6.44 ± 0.90 |

Feasibility for Mass Manufacturing: These calculations have been done for one operator with only one extruder and will work on one part at a time. Time taken for silicone mold printing is 1 minute with 2 minutes of heating time to cure the silicone. Epoxy printing takes 0.5 minutes with another 0.5 minutes taken by pick and place operation and it takes about 10 minutes to cure at 50° C. Conductive epoxy printing takes 1 minute with heating taking around 5 minutes. Printing of the asymmetric anchor takes about 0.5 minutes followed by 2 minutes of heating for curing.

Total manufacturing time: 21.5 minutes excluding Polyurethane and Parylene Coating which would be a batch type of manufacturing. This would result in 2.8 sensors being fabricated per hour. These parameters can be further optimized by changing the size and the quantity of epoxy being used which will determine the setting/curing time.

These calculations also do not take into consideration that some of these tasks can be done simultaneously with the use of buffer inventory. For example, silicone molds can be printed and cured while the epoxies are being cured with sensors in them. Maintaining a buffer inventory with batch type of fabrication at each step will help further improve the production as setup times would be reduced and utility of the 3D printer would improve. For instance, buffer can be provided between steps I and II (FIGS. 1A-B), III and IV (FIGS. 1C-D). Step I can be performed in batch, silicone can be cured and used as a buffer for step II. Step II and III have to be performed together and can be performed in batch. The batch manufactured after step III can be used as a buffer for step IV. Current setup time includes loading of syringes with different materials which takes around 2 minutes for each sensor. The bottleneck/limiting factor in this process is epoxy printing as epoxy cannot be loaded in syringe in large quantities due to the hardener present which starts the curing process the moment it is mixed with the epoxy. Continuous change of tips is typically required due to clogging.

Materials: Phosphate buffered saline (PBS), Pluronic F-127, and glycerol (G9012) were from Sigma Aldrich. Silicone (SI 595 CL) and Epoxy (EA 1C-LV) were from Loctite. De-ionized water (DIW) was obtained from a commercially available DIW system (Direct-Q 3UV; Millipore), poly(ethylene glycol) dimethylacrylate (PEGDMA) (750 Da), 2,2-dimethoxy-2-phenylacetophenone (DMPA), were purchased from Millipore Sigma. Lead zirconate titanate (PZT-5A; 72.4×72.4×0.127 mm) with nickel electrodes was purchased from Piezosystems (Woburn, MA). Polyurethane (Fast-Drying) was from Minwax. Silver Conductive Epoxy was from Atom Adhesives.

3D-Printing of Piezoelectric Cantilever Sensors: PZT sheets were diced into chips (5×1×0.127 mm³; American Dicing; Liverpool, NY). A cubical/cylindrical hollow mold was printed with silicone with a 20 gauge tip and cured on a heated printing platform. Epoxy was extruded inside the mold and PZT chip was placed in the center of the mold using the pick and place capability. A 27 gauge tip was used to extrude conductive epoxy connecting opposite sides of the distal end of the PZT to their respective copper tape connectors. The sensors were then coated with polyurethane and cured at room temperature. The 3D printing was done with a three-axis industrial dispensing robot (F5200N; Fisnar), and digital pressure regulator (Ultimus V; Nordson).

Hydrogel Preparation: Ink was prepared with 10 wt % solution of poly(ethylene glycol) dimethyacrylate (PEGDMA) (750 Da) in DIW, and 0.2% 2,2-Dimethoxy-2-phenylacetophenone (DMPA) from a 20% DMPA stock solution was used as a photoinitiator. PEGDMA hydrogels were cured with exposure to 365 nm UV light (1200 μW/cm² at 3 in.; UVGL-58).

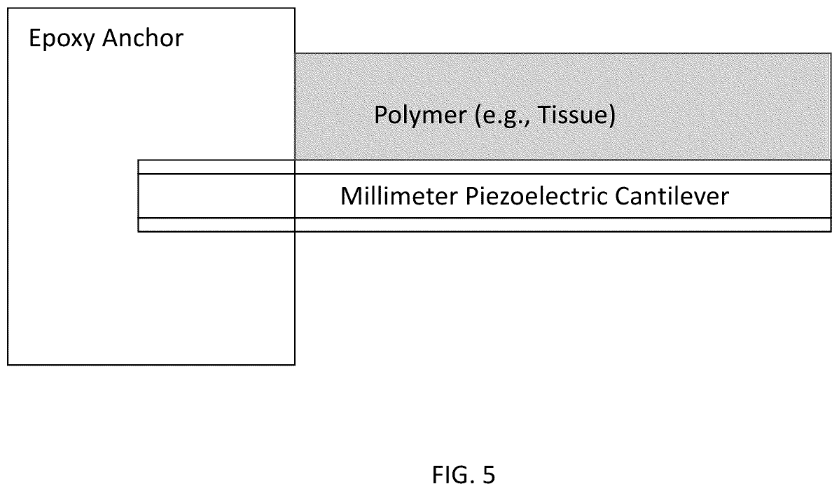

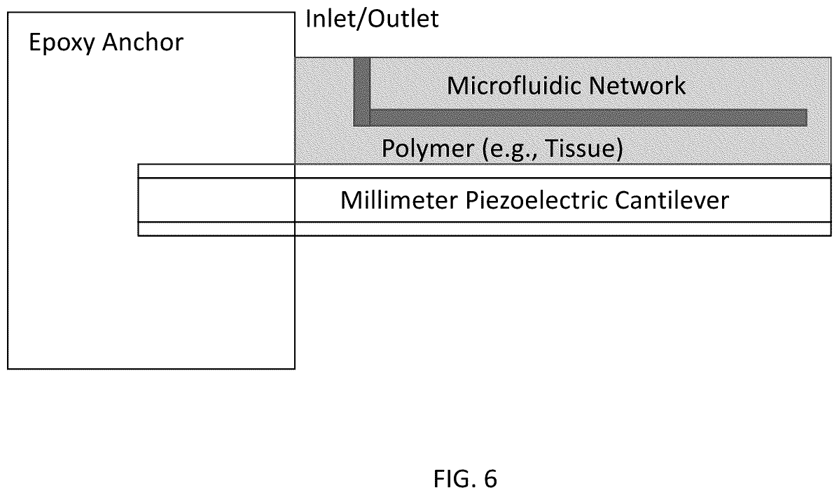

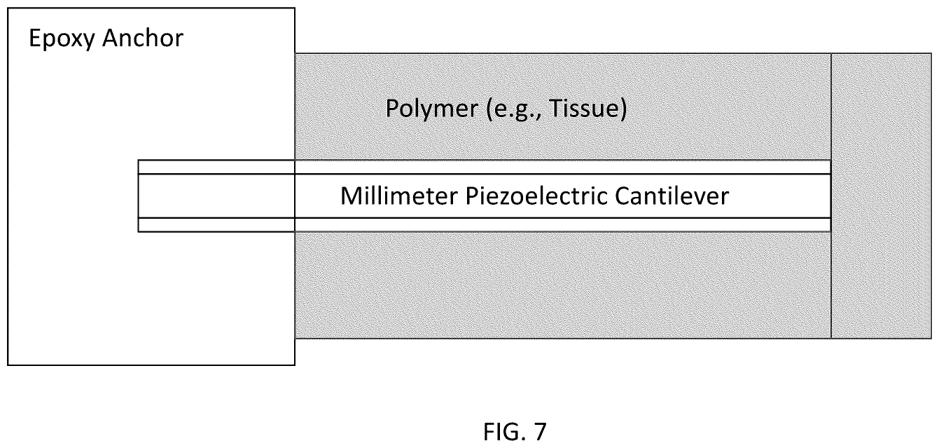

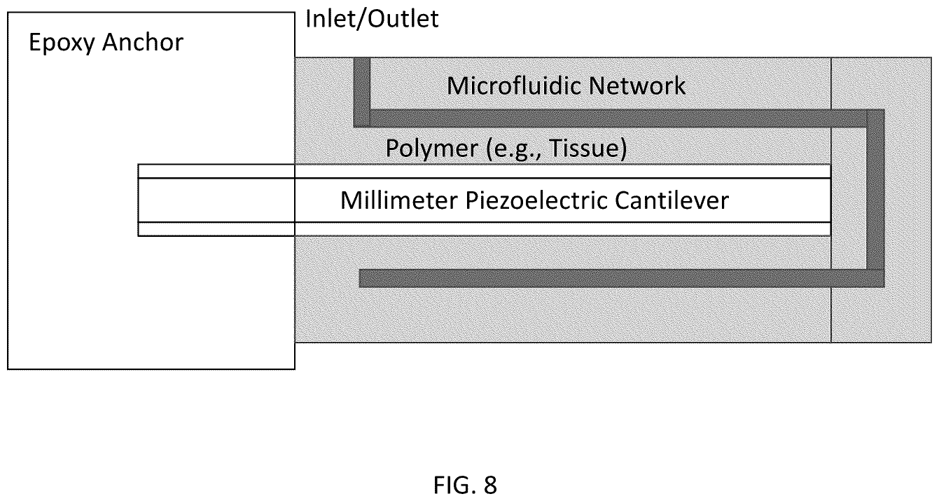

FIGS. 5-8 show piezoelectric cantilevers interacting with tissues, tissue models, or polymers according to embodiments of the invention. FIGS. 5-6 show embodiments in which the tissue model rests on top of the cantilever. FIGS. 7-8 show embodiments in which the cantilever is surrounded by the tissue model. FIGS. 6 and 8 show embodiments in which the tissue model comprises a microfluidic network.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references in this disclosure are each individually incorporated by reference herein in their entireties and as such are intended to provide an efficient way of supplementing the enabling disclosure of this invention as well as provide background detailing the level of ordinary skill in the art.

1. A method for production of a 3D printed piezoelectric cantilever sensor comprising:
3D printing of a sensor base/mold;
dispensing polymer within the sensor base/mold;
placing one or more piezoelectric transducer or uninsulated diced unimorph or bimorph piezoelectric chip with electrodes into the polymer to obtain a piezoelectric cantilever;
printing one or more conductive leads on one or more side/face of the piezoelectric cantilever;
printing an asymmetric anchor on one side/face of the piezoelectric cantilever, wherein the asymmetric anchor is the same or a different material than the polymer;
dispensing a coating on one or more of the sensor base/mold, piezoelectric cantilever and/or conductive leads; and
curing to provide a piezoelectric cantilever sensor.

2. The method of claim 1, further comprising connecting one or more of the conductive lead(s) to an electrically-conductive circuit.

3. (canceled)

4. The method of claim 1, wherein the piezoelectric cantilever sensor is configured to be capable of measuring within a frequency in the range of above 0 Hz to about 10 MHz.

5. The method of claim 1, wherein the curing is performed at a temperature ranging from about room temperature to 180° C.

6. The method of claim 5, wherein the curing is performed for a period of about 1 minute to about 3 hours.

7. The method of claim 6, further comprising:
placing and/or securing one or more of the piezoelectric cantilever sensors into a well of a well plate.

8. The method of claim 7, wherein the piezoelectric cantilever sensor is constructed in the well plate.

9. A method comprising:
providing one or more piezoelectric cantilever sensor;
dispensing the piezoelectric cantilever sensor in a well plate; and
dispensing one or more o substance in the well plate.

10. The method of claim 9, wherein:
tissue or a tumor models are dispensed in the well plate; and
at least part or all of the piezoelectric cantilever sensor is embedded in the tissue or tumor; and
further comprising measuring one or more mechanical properties of the tissue or tumor model with the piezoelectric cantilever sensor.

11. The method of claim 10, wherein one or more of the mechanical properties are measured using one or more electrical impedance measurements.

12. The method of claim 10, wherein one or more of the mechanical properties comprises stiffness of the tissue or tumor model and/or mass changes in the tissue, the tumor model and/or in cell culturing media or tissue engineering media.

13. The method of claim 12, further comprising measuring the stiffness and/or mass change of the tissue or tumor model in response to one or more tissue or tumor model treatment.

14. The method of claim 9, wherein the one or more additional substance is one or more tissue or tumor model, and further comprising monitoring one or more of cell motility, mitosis, cell growth, and/or motion and/or cell culture media density and/or viscosity.

15. A device comprising:
a well plate or cavity for tissue culture with one or more wells/cavities; and
a piezoelectric cantilever sensor disposed in or capable of being placed in one or more of the wells/cavities.

16. (canceled)

17. The device of claim 15, wherein the piezoelectric cantilever sensor comprises a piezoelectric cantilever/chip with an asymmetric anchor on one face of the cantilever/chip.

18. The device of claim 17, wherein the cantilever/chip has a length of about 1-5 mm.

19. A kit comprising the device of claim 15 further comprising:
cells and/or engineered tissue,
3D printable and/or 3D printed cells, tissue and/or bioink, and/or
cell culturing media and/or tissue engineering media.

20. The method of claim 9, further comprising:
measuring mass or concentration of one or more analyte in the additional substance.

21. The method of claim 20, wherein the one or more substance is a solution or crosslinked matrix or a gel.

22. (canceled)

23. The method of claim 1, wherein the dispensing comprises dispensing polymer within the sensor base/mold and surrounding chamber.
